(19) United States
(12) Patent Application Publication
(10) Pub. No.: US 2017/0086650 A1
(43) Pub. Date: Mar. 30, 2017

(54) Adapter Device for Securing an Eyepiece to a Mobile Phone

(71) Applicant: Save My Scope Inc., Sleepy Hollow, IL (US)

(72) Inventors: Hasnain Valika, Houston, TX (US); Murtaza Valika, Sleepy Hollow, IL (US); Taher Valika, Chicago, IL (US)

(73) Assignee: Save My Scope Inc.

(21) Appl. No.: 15/276,738

(22) Filed: Sep. 26, 2016

Related U.S. Application Data
(60) Provisional application No. 62/233,060, filed on Sep. 25, 2015.

(51) Int. Cl. A61B 1/00; H04M 1/02

(52) U.S. Cl. CPC A61B 1/0014; H04M 1/0264; H04B 1/3888

(57) ABSTRACT

A housing for an adapter device to secure an eyepiece to a phone case includes a body including a cavity with a spring housing extending therefrom, a retaining member within the body, and a spring positioned within the spring housing of the body and biased against the retaining member. The retaining member is configured to capture the eyepiece within the cavity.

### CROSS-REFERENCE TO RELATED APPLICATION

This application claims the benefit of priority to U.S. Provisional Application No. 62/233,060 filed on Sep. 25, 2015, the disclosure of which is incorporated herein by reference.

### BACKGROUND OF THE INVENTION

The present invention relates to a device for securing an endoscope to a mobile phone in order to utilize the camera function of the phone. Specifically, the present invention provides a device having a housing with a retaining member that receives and secures an eyepiece of an endoscope within a cavity of the housing.

Endoscopy refers to a medical procedure of viewing internal organs of the body using an endoscope. A conventional endoscope includes a long tube with a camera mounted on a first end and an eyepiece secured to a second end. During an endoscopy procedure, a user inserts the first end of the tube into a cavity of a body and views the interior of the body through the eyepiece at the second end.

Use of the eyepiece can be awkward and limiting for the user. Physicians often mount the eyepiece onto a video tower equipped with a screen to enlarge the view of the image and allow for further magnifying. A medical team can then view the imaging of the examination together, rather than requiring multiple examinations by individual members of the team.

A mobile phone may also be used to enlarge the image seen through the eyepiece. The camera lens of the mobile phone may capture the image viewed through the eyepiece, and the image is then shown on the screen of the mobile phone. The mobile phone acts as a viewfinder for the endoscope, enlarging the view and allowing the physician and medical team to view the examination at the same time. Mobile phones such as the Apple® iPhone® 5/5s/6 provide image quality that matches the high-resolution imaging available through standard video equipment. Further, mobile phones are more convenient and require less space than video equipment.

To use a mobile phone as a viewfinder, an adapter secures the eyepiece to a mobile phone. Current adapters on the market require an assembly of parts to mount the eyepiece of the endoscope onto the mobile phone. Adapters that are designed for use with a variety of mobile phones involve the assembly of specific parts for use with specific mobile phones. These adapters are cumbersome and time-consuming to use, particularly when performing the endoscopy.

Accordingly, there is a need for an adapter device to connect a mobile phone to an eyepiece of an endoscope easily and efficiently.

### SUMMARY OF THE INVENTION

To meet the needs described above and others, the present disclosure provides an adapter device including a housing with a retaining member that holds the eyepiece of an endoscope within the housing. During use, the user can easily attach the mobile phone to the endoscope by moving the handle of the retaining member, inserting the eyepiece into the housing, and releasing the handle.

The adapter device of the present application includes a housing including a body including a cavity with a spring housing extending therefrom, a retaining member within the body, and a spring positioned within the spring housing of the body and biased against the retaining member. The retaining member is configured to capture the eyepiece within the cavity.

A cover with an opening to receive the eyepiece may be formed integrally with the body or may be provided as a separate component to be attached to the body with fasteners.

The body further includes a lower portion, a side wall adjacent an edge of the lower portion, and an upper portion. The retaining member is positioned along the side wall between the lower portion and the cover. The side wall includes an opening, and the retaining member includes a handle that extends through the opening of the side wall.

The upper portion of the body includes an angled surface that forms a point with the side wall. The retaining member has an end that complements the angled surface of the upper portion of the body. In further embodiments, the retaining member has an arcuate shape that complements a cylindrical shape of the side wall of the body, and the retaining member rotates within the cavity about a fastener.

In another embodiment, the lower portion includes an inner surface, a projection extending from the inner surface, and a groove indented within the inner surface spaced from the projection.

In a further embodiment, an adapter device to secure an eyepiece to a phone case includes a phone case with an aperture and a housing secured to the phone case including a cavity adjacent to the aperture. The housing includes a body including a cavity with a spring housing extending therefrom, a retaining member within the cavity of the body, and a spring positioned within the spring housing of the body and biased against the retaining member. The retaining member is configured to capture the eyepiece within the cavity. In a further embodiment, the eyepiece is captured within the adapter device aligns with the aperture of the phone case.

Standard adapters requiring the assembly of multiple components are particularly time-consuming in an environment where time is extremely valuable. While standard adapters require parts to be removed and added in order to secure the eyepiece within the adapter, operation the adapter device of the present invention can be accomplished with only one hand. The adapter device requires only movement of a handle to increase the size of the opening, and the release of the handle captures the eyepiece within the cavity of the adapter.

One advantage of the present application is the convenience and efficiency of securing the eyepiece of an endoscope to a mobile phone.

Another advantage is providing a portable viewfinder to enlarge the image of the eyepiece of an endoscope.

A further advantage is eliminating the need for large viewing equipment such as a video tower during an endoscopy procedure.

Another advantage is eliminating the need to perform multiple examinations by individual members of a medical team.

Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations described herein.

### BRIEF DESCRIPTION OF THE DRAWINGS

The drawing figures depict one or more implementations in accord with the present concepts, by way of example only, not by way of limitations. In the figures, like reference numerals refer to the same or similar elements.

FIG. 1 is an isometric view of an adapter device of the present application retaining the eyepiece of an endoscope.

FIG. 2 is an isometric view from the front of the adapter device of FIG. 1.

FIG. 3 is an isometric view from the back of the adapter device of FIG. 1.

FIG. 4 is an exploded view of the adapter device of FIG. 1.

FIG. 5 is a sectional view generally taken along lines 5-5 of FIG. 2.

FIG. 6 is a plan view of the adapter device of FIG. 1.

FIG. 7 is a plan view of the adapter device of FIG. 1 with the cover removed in the resting position.

FIG. 8 is a side elevation view of the adapter device of FIG. 1 with the cover removed in the active position.

### DETAILED DESCRIPTION

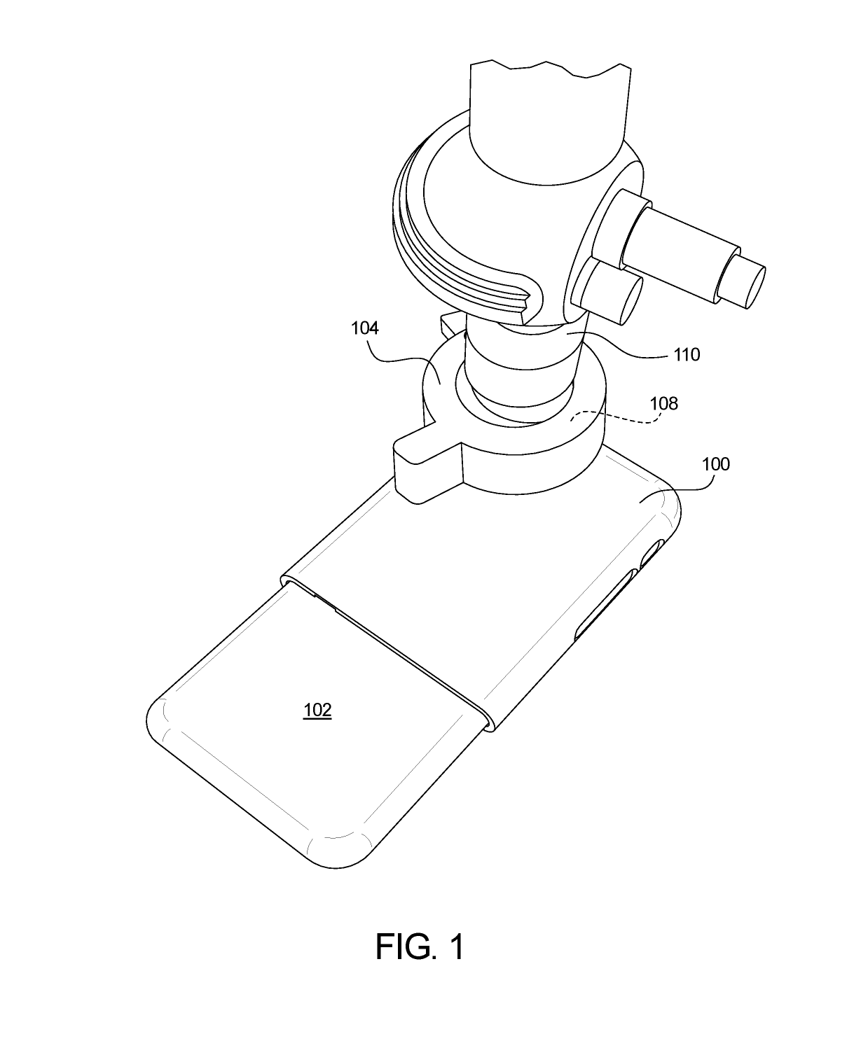

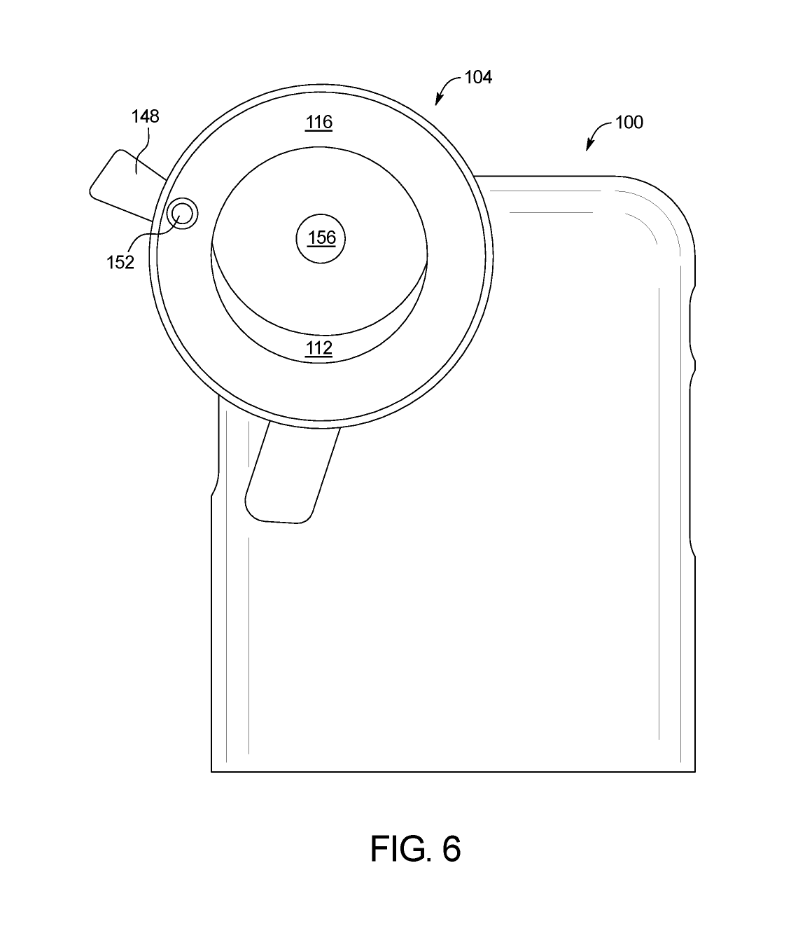

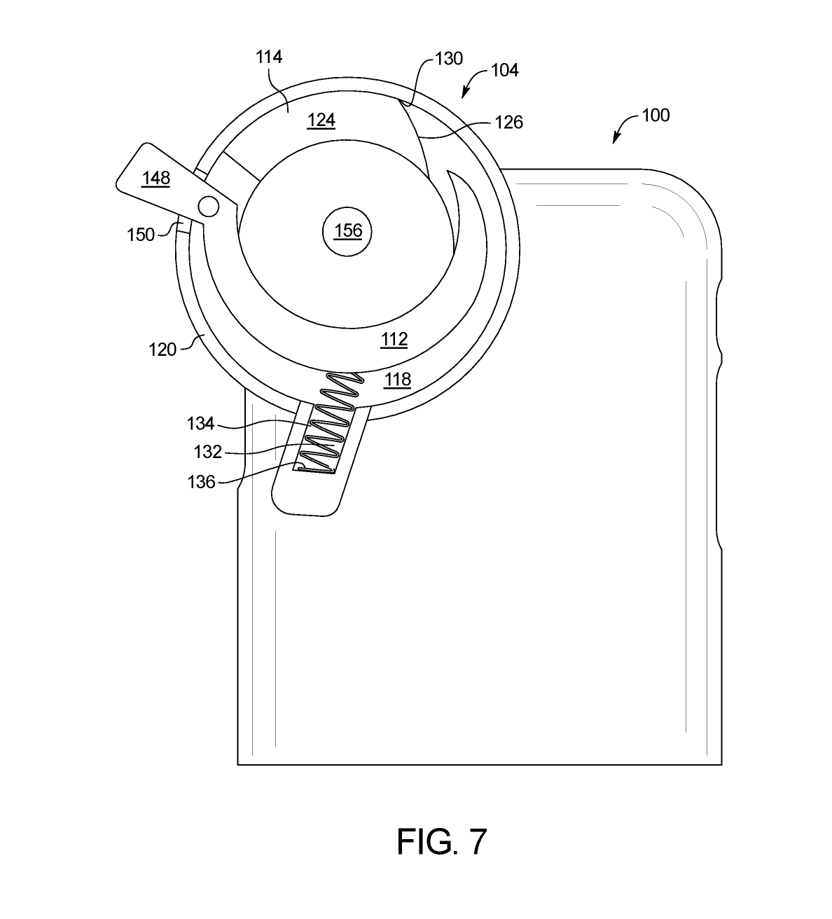

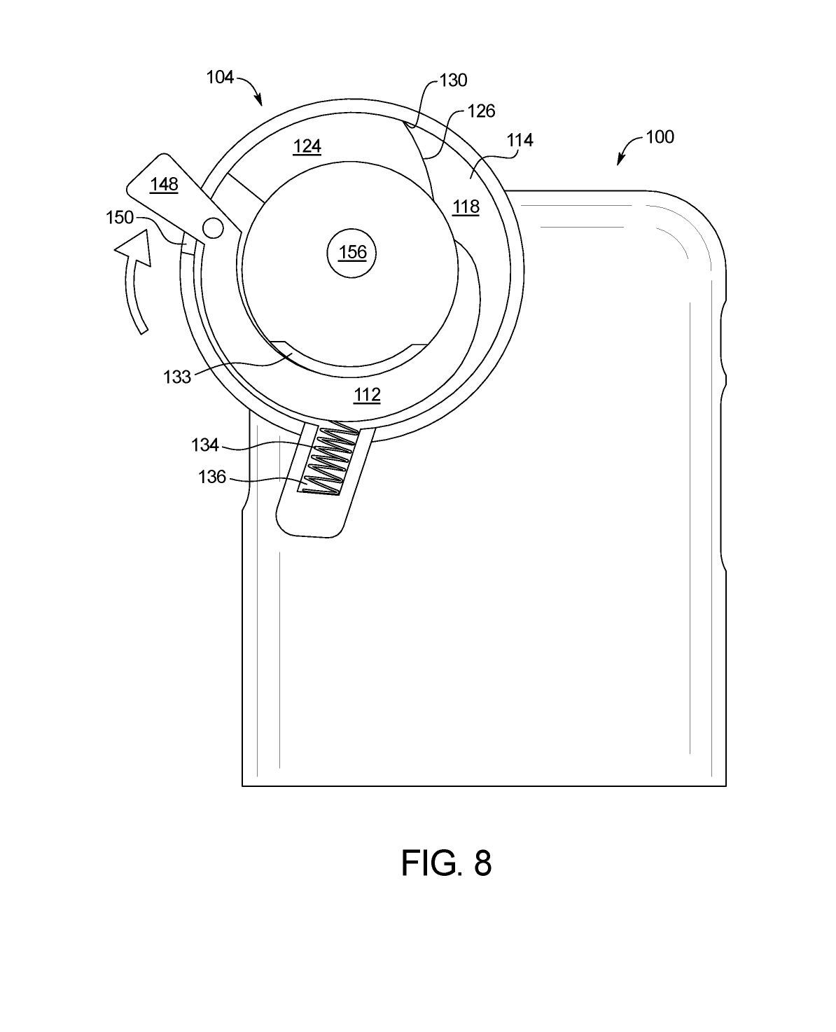

As illustrated in the FIG. 1, the adapter device 100 of the present application mounts onto a mobile phone 102 and includes a housing 104 with a cavity 106 that receives and holds an eyepiece 108 of an endoscope 110. Referring to FIGS. 2-8, a spring-loaded retaining member 112 within the housing 104 is moveable between a resting position of FIG. 6 and an active position of FIG. 7 to capture the eyepiece 108 within the cavity 106.

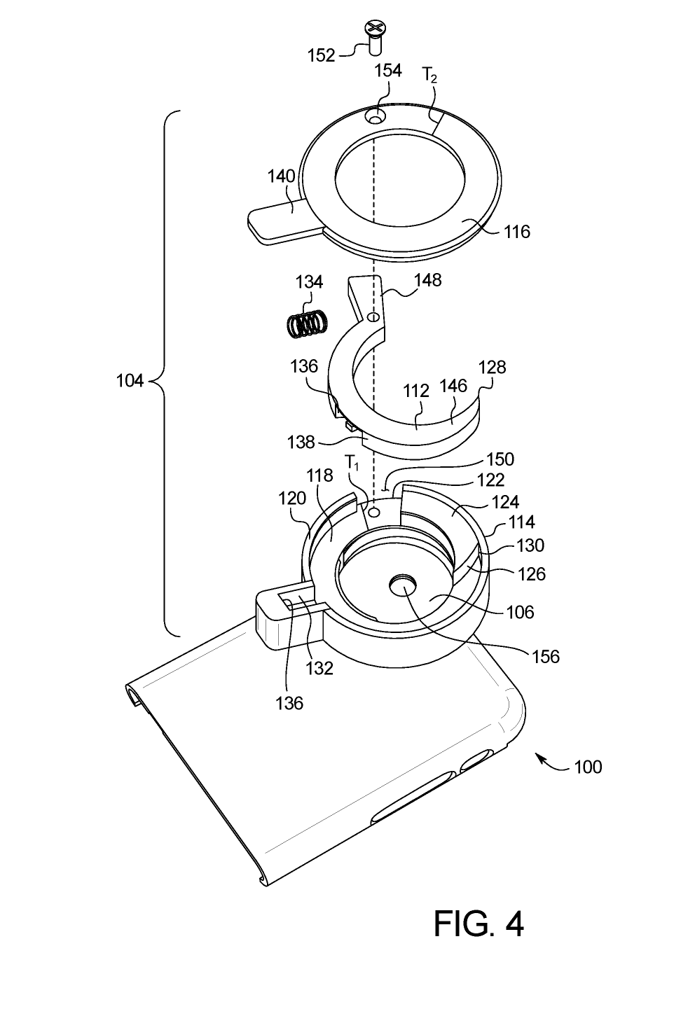

Referring to FIG. 4, the housing 104 includes a body 114 shaped to hold the retaining member 112. A cover 116 of the body 114 encloses the retaining member 112 against the body 114. In the illustrated embodiment, the cover 116 is formed integrally with the body 114. FIG. 4 shows the cover 116 as a separate component from the body 114 in order to illustrate the nesting of the retaining member 112 within the body 114. In other embodiments, the cover 116 may be separate from the body 114 and secured thereto with one or more fasteners. Further, the cover 116 includes an opening for receiving the eyepiece as shown in FIG. 1.

The cavity 106 is defined by the body 114 and the retaining member 112. Specifically, an annular lower portion 118 of the body 114 having a thickness T1 provides a base against which the retaining member 112 is maintained. A cylindrical side surface 120 extends along an outer edge 122 of the lower portion 118, and an upper portion 124 having an arcuate shape is positioned adjacent to the lower portion 118 and the cylindrical side surface 120. The upper portion 124 includes an angled surface 126 that complements an end 128 of the retaining member 112 and forms a point 130 with the cylindrical side surface 120.

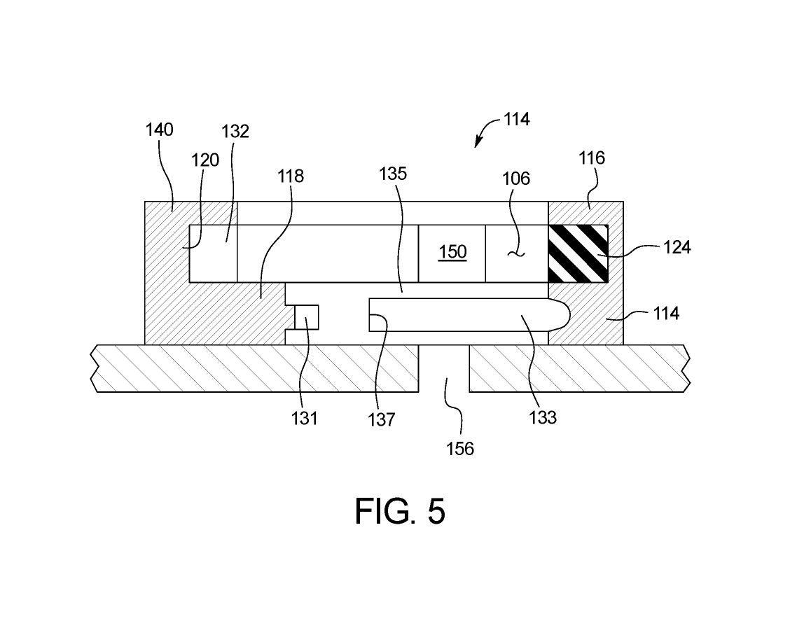

Additionally, the lower portion 118 of the body 114 includes a projection 131 and an indented groove 133 along portions of the inner surface 135 in order to more securely and snugly hold the eyepiece 108 within the cavity 106. As shown in FIGS. 4, 5, and 8, the projection 131 extends inwardly into the cavity 106 from the inner surface 135 of the lower portion 118. Referring to FIGS. 4 and 5, the groove 133 is formed along the inner surface 135 of the lower portion 118 spaced from the projection 131. A depth of the groove 133 increases gradually from outer ends 137 towards the center of the groove 133. In other embodiments, the depth, shape, and length of the projection 131 and/or the groove 133 may vary or be consistent.

The body 114 also includes a spring opening 132 that houses a spring 134. The spring 134 is biased against an end wall 136 of the spring opening 132 and a notch 136 within a side surface 138 of the retaining member 112. In other embodiment, the spring 134 may be biased directly against the side surface 138 of the retaining member 112. Other shapes and types of springs may be used in the adapter device 100 of the present application.

The cover 116 of the body 114 has an annular shape with an extension 140 to cover the spring opening 132. In the illustrated embodiment, the thickness T2 of the annular shape is similar to the thickness T1 of the lower portion 118 of the body 114. In embodiments where the cover 116 is separate from the remainder of the body 114, one or more fasteners such as screws extend through openings within the cover 116 into the body 114 of the housing 104.

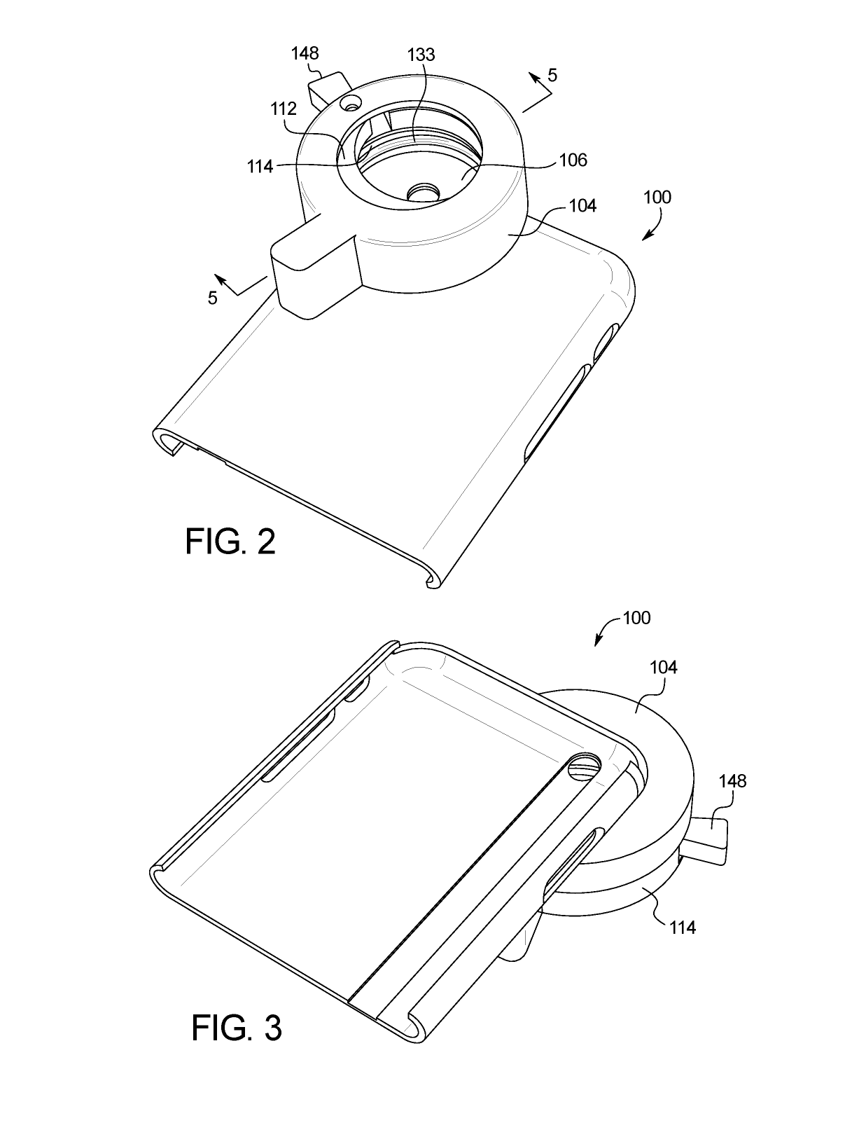

As seen in FIG. 3, the retaining member 112 has a curved retaining body 146 that complements the cylindrical wall 120 of the body 114 of the housing 104. A handle 148 extends outwardly from the retaining body 146 and through a handle opening 150 of the cylindrical wall 120 of the body 114. A fastener 152 such as a screw extends through an opening 154 within the cover 116 of the body 114, through the handle 148 of the retaining member 112, and into the lower portion 118 of the body 114. The retaining member 112 rotates about the third fastener 152 upon movement of the handle 148.

During use, the spring 134 within the spring opening 132 biases the retaining member 112 so that, in the resting position of FIG. 7, the end 128 of the retaining member 112 fits into or adjacent to the point 130 formed by the upper portion 124 and the cylindrical wall 120 of the body 114. The user moves the handle 148 of the retaining member 112 against the force of the spring 134 in the active position of FIG. 8. The user then inserts the eyepiece 108 into the cavity 106, positions the eyepiece 108 within the groove 133 of the body 114, and releases the handle 148 so that the retaining member 112 holds the eyepiece 108 within the housing 104.

The adapter device includes an aperture 156 adjacent to the cavity 106 of the housing 104. In one embedment, the aperture 156 is approximately centered about the camera lens of the mobile phone 102 when the adapter device 100 is mounted on the mobile phone 102. so that the camera lens captures the image of the eyepiece 108 of the endoscope 110 when mounted in the adapter device 100.

It should be noted that various changes and modifications to the presently preferred embodiments described herein will be apparent to those skilled in the art. Such changes and modifications may be made without departing from the spirit and scope of the present invention and without diminishing its attendant advantages.

We claim:

1. A housing for an adapter device to secure an eyepiece to a phone case, the housing comprising:
a body including a cavity with a spring housing extending therefrom;
a retaining member within the body; and
a spring positioned within the spring housing of the body and biased against the retaining member;
wherein the retaining member is configured to capture the eyepiece within the cavity.

2. The housing of claim 1, wherein the housing further includes a cover with an opening for receiving the eyepiece.

3. The housing of claim 2, wherein the cover is formed integrally with the body.

4. The housing of claim 1, wherein the body includes a lower portion, a side wall adjacent an edge of the lower portion, and an upper portion.

5. The housing of claim 4, wherein the retaining member is positioned along the side wall between the lower portion of the body and the cover.

6. The housing of claim 5, wherein the side wall includes an opening, and wherein the retaining member includes a handle that extends through the opening of the side wall.

7. The housing of claim 4, wherein the upper portion of the body includes an angled surface that forms a point with the side wall.

8. The housing of claim 7, wherein the retaining member has an end that complements the angled surface of the upper portion of the body.

9. The housing of claim 4, wherein the retaining member has an arcuate shape that complements a cylindrical shape of the side wall of the body.

10. The housing of claim 9, wherein the retaining member rotates within the cavity about a fastener.

11. The housing of claim 4, wherein the lower portion includes an inner surface, a projection extending from the inner surface, and a groove indented within the inner surface spaced from the projection.

12. An adapter device to secure an eyepiece to a phone case, the adapter device comprising:
a phone case including an aperture; and
a housing secured to the phone case including a cavity adjacent to the aperture, the housing including:
a body including a cavity with a spring housing extending therefrom;
a retaining member within the cavity of the body; and
a spring positioned within the spring housing of the body and biased against the retaining member;
wherein the retaining member is configured to capture the eyepiece within the cavity.

13. The adapter device of claim 12, wherein the eyepiece captured within the adapter device aligns with the aperture of the phone case.

14. The adapter device of claim 12, wherein the retaining member has an arcuate shape that complements the cylindrical shape of the body.

15. The adapter device of claim 12, wherein the housing further includes a fastener that extends through the body and the retaining member, and wherein the retaining member rotates about the fastener.
